(19) United States
(12) Patent Application Publication
(10) Pub. No.: US 2009/0130171 A1
(43) Pub. Date: May 21, 2009

(54) High concentration gentian violet containing medical devices and methods of making same

(76) Inventors: Hiep Do, Sinking Spring, PA (US); Daniel J. Spangler, Wernersville, PA (US); Joel Rosenblatt, Pottstown, PA (US); Barrett Remington, Hamburg, PA (US); Onajite Okoh, Reading, PA (US)

Correspondence Address: AMSTER, ROTHSTEIN & EBENSTEIN LLP, 90 PARK AVENUE, NEW YORK, NY, 10016, US

(21) Appl. No.: 12/286,791

(22) Filed: Oct. 2, 2008

Related U.S. Application Data
(60) Provisional application No. 60/997,490, filed on Oct. 3, 2007.

(51) Int. Cl. A61F 2/04; A61K 31/136

(52) U.S. Cl. 424/423; 514/648

(57) ABSTRACT

Implantable medical devices, such as catheters, treated with a high concentration of gentian violet, and methods of preparing these medical devices are provided.

### CROSS-REFERENCE TO RELATED APPLICATION

This application claims the benefit of U.S. Provisional Patent Application No. 60/997,490, filed on Oct. 3, 2007, the content of which is hereby incorporated by reference into the subject application.

### FIELD OF THE INVENTION

The present invention relates to implantable medical devices, in particular catheters, treated with a high concentration of gentian violet and to methods of preparing these medical devices.

### BACKGROUND OF THE INVENTION

Various publications are referred to throughout this application. Full citations for these references may be found at the end of the specification immediately preceding the claims. The disclosures of these publications are hereby incorporated by reference in their entireties into the subject application to more fully describe the art to which the subject application pertains.

Implantable medical devices such as tunneled catheters play a major role in general medicine. Aside from pneumothorax and hemorrhage-like complications which are associated with their initial insertion, catheters are associated with the long-term risk of infection (e.g., Harter et al. 2002; Rooden et al. 2005; Safdar and Maki 2003; Saint et al. 2000). Colonization of microorganisms on the surfaces of such devices following implantation can produce serious and costly complications, including the need to remove and/or replace the implanted device and/or vigorous treatment of secondary infections. To minimize the risk of infection, implantable medical devices can be treated with gentian violet (GV) or other antimicrobial agents (e.g. Hanna et al. 2006; U.S. Pat. Nos. 5,451,424, 5,688,516, 5,707,366, 6,261,271, 6,273,875, 6,528,107).

U.S. Pat. No. 5,709,672 describes a solvent impregnation method of gentian violet at very low concentration loadings, from 0.01% to less than 1%, and preferably from 0.01% to 0.1%. U.S. Patent Application publications 2003/0078242 A1, 2005/0131356 A1 and 2005/0197634 A1 also teach solvent impregnation for dyes such as gentian violet. These methods involve multiple steps of first forming the medical device and then subsequent treatment to coat or impregnate the device with an agent such as gentian violet.

### SUMMARY OF THE INVENTION

The present invention provides implantable medical devices, in particular catheters, treated with gentian violet, wherein the device comprises a thermoplastic treated with gentian violet or a thermoset treated with gentian violet, wherein the concentration of gentian violet in the device is greater than 1% by weight of the thermoplastic or the thermoset.

The invention also provides laminated implantable medical devices, such as catheters, comprising a thermoplastic layer treated with gentian violet or a thermoset layer treated with gentian violet, wherein the concentration of gentian violet in the layer is greater than 1% by weight of the thermoplastic in the layer or the thermoset in the layer.

The invention further provides implantable medical devices, such as catheters, comprising thermoplastic treated with gentian violet, wherein the thermoplastic has a processing temperature below 207° C. and wherein the concentration of gentian violet in the device is 0.05% to 20% by weight of the thermoplastic.

The invention also provides laminated implantable medical devices, such as catheters, comprising a thermoplastic layer treated with gentian violet, wherein the thermoplastic in the layer has a processing temperature below 207° C. and wherein the concentration of gentian violet in the layer is 0.05% to 20% by weight of the thermoplastic in the layer.

The invention further provides methods of preparing implantable medical devices treated with gentian violet, where the methods involve extruding gentian violet with a thermoplastic at a temperature below 207° C., where the concentration of gentian violet is 0.05% to 20% by weight of the thermoplastic.

The invention further provides kits comprising implantable medical devices, in particular catheters, treated with gentian violet; and non-ionized metals, and optionally peroxide, for use in decoloring a gentian violet stain produced by the device.

### BRIEF DESCRIPTION OF THE DRAWINGS

FIG. 1. HPLC chromatogram of 50 μg/mL gentian violet (GV) (ScienceLab)/200 μg/mL CHA standard in water/ACN/MeOH @ 588 nm.

FIG. 2. HPLC chromatogram of gentian violet compounded in Tecothane® 2095A @ 588 nm.

FIG. 3. HPLC chromatogram of gentian violet Standard (Aldrich) @ 588 nm.

FIG. 4. HPLC chromatogram of gentian violet from Tecothane® 2095A Tube sample @ 588 nm.

FIG. 5. Chromatogram at 588 nm of Tecothane® (w/30% BiOCl) compounded with 0.5% GV (Aldrich ACS grade) at 170° C.

FIG. 6. Chromatogram at 588 nm of degraded Tecothane® (w/30% BiOCl) compounded with 0.5% GV (Aldrich ACS grade) at 217° C.

FIG. 7. Chromatogram at 253 nm of Tecothane® (w/30% BiOCl) compounded with 0.5% GV (Aldrich ACS grade) at 170° C.

FIG. 8. Chromatogram at 253 nm of degraded Tecothane® (w/30% BiOCl) compounded with 0.5% GV (Aldrich ACS grade) at 217° C.

FIG. 9. Chromatogram at 280 nm of Tecothane® (w/30% BiOCl) compounded with 0.5% GV (Aldrich ACS grade) at 170° C. 5.896 min peak is due to solvent.

FIG. 10. Chromatogram at 280 nm of degraded Tecothane® (w/30% BiOCl) compounded with 0.5% GV (Aldrich ACS grade) at 217° C. 5.974 min peak is due to solvent.

FIG. 11A-11B. Removal of topical gentian violet (GV) stain on plated agar by silver activated hydrogen peroxide. A) 10 μl 0.1% GV+100 μl H₂O₂; B) 10 μl 0.1% GV+100 μl H₂O₂+0.00867 g Ag.

### DETAILED DESCRIPTION OF THE INVENTION

The invention provides an implantable medical device treated with gentian violet, wherein the device comprises a thermoplastic treated with gentian violet or a thermoset treated with gentian violet, wherein the concentration of gentian violet in the device is greater than 1% by weight of the thermoplastic or the thermoset.

The invention also provides a laminated implantable medical device comprising a thermoplastic layer treated with gentian violet or a thermoset layer treated with gentian violet, wherein the concentration of gentian violet in the layer is greater than 1% by weight of the thermoplastic in the layer or the thermoset in the layer.

The invention further provides an implantable medical device comprising thermoplastic treated with gentian violet, wherein the thermoplastic has a processing temperature below 207° C. and wherein the concentration of gentian violet in the device is 0.05% to 20% by weight of the thermoplastic.

The invention also provides a laminated implantable medical devices comprising a thermoplastic layer treated with gentian violet, wherein the thermoplastic in the layer has a processing temperature below 207° C. and wherein the concentration of gentian violet in the layer is 0.05% to 20% by weight of the thermoplastic in the layer.

As used herein, a “thermoplastic” is a polymeric material that can be reversibly changed from a viscous and deformable (processable) material to a formed (shaped) solid material by heating and cooling. As used herein, a “thermoset” is a polymeric material that must be irreversibly crosslinked into a 3-dimensional network in order to produce a formed (shaped) solid material.

Examples of thermoplastics that can be used in the present invention include, but are not limited to, polyurethanes (such as one or more of Tecothane®, Carbothane®, Tecoflex®, Tecophilic®, Texin®, and Pellethane®), polyolefins, vinyl polymers (such as vinyl chloride, butyl rubbers, ethylene vinyl acetate and copolymers), acrylate polymers, polyamides, polyesters, fluoroelastomers (such as hexafluoropropylene vinylidine fluoride copolymers) and copolymers (including block copolymers and segmented block copolymers) and blends thereof. Polyurethane is a preferred thermoplastic.

Examples of thermosets include, but are not limited to, silicones, vulcanized rubbers, polyimides and epoxies.

Preferably, the thermoplastic and the thermoset are biocompatible.

In different examples, the concentration of gentian violet in the device is greater than 1.1%, 1.5%, 2%, 2.5%, 3%, 4%, 5%, 7.5% or 10% by weight of the thermoplastic or the thermoset. The concentration of gentian violet can be, for example, up to 20% by weight of the thermoplastic or the thermoset. Where the device is a laminated device, the concentration of gentian violet in the layer containing gentian violet may be, for example, greater than 1% of the weight of the thermoplastic or the thermoset in the layer; however, the total device concentration of gentian violet may be less than 1% by weight depending on the size of the gentian violet layer relative to the rest of the laminated device.

The device can also further comprise, for example, of one or more of silicone elastomers, thermoplastics, fluoropolymers, polyurethanes, polyolefins, vinyl polymers (such as vinyl chloride, butyl rubbers, ethylene vinyl acetate and copolymers), acrylate polymers, polyamides, polyesters, fluoroelastomers (such as hexafluoropropylene vinylidine fluoride copolymers), and copolymers (including block copolymers and segmented block copolymers) and blends thereof.

Methods of processing gentian violet include, for example, extrusion, injection molding, blow molding, compression molding, or any other hot melt processes. Preferably, the gentian violet is introduced by extrusion. Prior to extrusion, the thermoplastic resin can be precoated with gentian violet or the thermoplastic resin can be compounded with gentian violet powder. Preferably, the thermoplastic used in the extrusion process has a processing temperature below 207° C.

Where the device comprises a thermoset, since thermosets are not melt processed, gentian violet can be introduced by solution impregnation into the thermoset or gentian violet can be incorporated by a direct blending process into the uncured thermoset resin so long as the resin and gentian violet remain stable through the curing process.

Laminated medical devices can be produced by, for example, coextrusion, insert molding or sequential coating.

The invention also provides a method of preparing an implantable medical device treated with gentian violet, where the method comprises extruding gentian violet with a thermoplastic at a temperature below 207° C., where the concentration of gentian violet is 0.05% to 20% by weight of the thermoplastic. The implantable medical device is formed from the gentian violet-treated thermoplastic. Preferably, the concentration of gentian violet is greater than 1% by weight of the thermoplastic. The processing temperature can be, for example, below 200° C.

The thermoplastic used in this method is preferably a low processing temperature thermoplastic, such as for example one or more of a polyurethane (such as one or more of Tecothane®, Carbothane®, Tecoflex®, Tecophilic®, Texin® and Pellethane®), a polyolefin, a vinyl polymer (such as vinyl chloride, butyl rubber, ethylene vinyl acetate and copolymers), an acrylate polymer, a polyamide, a polyester, a fluoroelastomer (such as hexafluoropropylene vinylidine fluoride copolymer) and copolymers (including block copolymers and segmented block copolymers) and blends thereof. Polyurethane is a preferred thermoplastic. Thermoplastics that require processing temperatures above the temperature at which degradation of gentian violet occurs are not suitable for this method unless the thermoplastic is treated, e.g. by plasticization, to lower its processing temperature below the temperature at which gentian violet degrades. Examples of polyurethanes having processing temperatures above 207° C. include: ISOPLAST® 2510 (Dow), manufacturer recommended melt temperature 220-245° C.; ISOPLAST® 101 LGF 40 NAT (Dow), manufacturer recommended melt temperature 240-260° C.; ISOPLAST® 202 EZ (Dow), manufacturer recommended melt temperature 240-260° C.; ISOPLAST® 2531 (Dow), manufacturer recommended melt temperature 230-250° C.; ISOPLAST® 301 (Dow), manufacturer recommended melt temperature 230-250° C.; and ISOPLAST® 302 EZ (Dow), manufacturer recommended melt temperature 238-260° C.

Prior to extrusion, the thermoplastic resin can be precoated with gentian violet or the thermoplastic resin can be compounded with gentian violet powder.

The present invention has the advantage that, in contrast to prior art methods, it uses a single step compounding and extrusion/molding versus multiple steps of prior art methods of first forming the device and subsequent treatment with agents, and can thus be more cost effective. An additional advantage of the present invention is that gentian violet can be incorporated at much higher concentrations than anticipated in the prior art using this method, which provides for much longer durations of antimicrobial protection.

Other agents that might not be stable in combination with gentian violet can be coated as a separate layer (either through coextrusion or through surface coating or impregnation) and thereby provide enhanced antimicrobial protection over that provided by gentian violet alone.

Additional antimicrobial agents include, for example, biguanides (including chlorhexidine, alexidine, PHMB—polyhexamethylbiguanide), antifungals (including azole and azole derivatives, rapamycin), phenolic antiseptics (e.g., triclosan), disulfuram, antimicrobial dyes (including methyl violet, methylene blue), cationic steroids, antimicrobial metal ions salts or conjugates (e.g., silver, silver sulfadiazine, zinc, copper, bismuth, gallium), biofilm inhibitors (including bismuth thiols, RIP (RNA III inhibiting peptide), furanones and their conjugates or derivatives, inhibitors of autoinducer 2, its kinases or its receptors, inhibitors of homoserine lactones, its kinases or its receptors), antibiotics and combinations (including tetracyclines, clindamycin, rifamycins, aminoglycosides, penicillins, cephalosporins, quinolones and fluoroquinolones, macrolides, carbapenems, peroxides, peroxyacids, glycopeptides (e.g., vancomycin, teicoplanin), polypeptides (e.g., bacitracin, polymixin B), sulfonamides, muciprocin, linezolid, chloramphenicol), chemotherapeutic agents (including DNA alkylating agents, mitomycin C, adriamycin, bleomycin, 5 fluorouracil, taurolidine, cisplatins, carboplatins), antimicrobial host defense proteins, host defense protein mimetics, their fragments and conjugates and salts thereof, and organic alcohols such as, for example, ethanol, isopropanol and benzyl alcohol.

The device can comprise a plurality of different regions, and gentian violet can be present in less than all of the regions. For example, if the device is a catheter, the catheter can comprise a tip region where gentian violet is present in the tip region. As another example, the catheter can comprise a proximal hub region where gentian violet is present in the hub region.

The invention also provides implantable medical devices prepared by any of the methods disclosed herein.

The implantable medical device can be, for example, a catheter such as a catheter for implantation in a vessel such as a blood vessel or in a body cavity in a subject. Examples of such catheters include transcutaneous catheters; vascular catheters including peripheral catheters, central catheters, venous catheters, and arterial catheters; urinary catheters; tracheal catheters or tubes; dialysis catheters; and catheters used for local delivery of anesthetics or other drugs (such as chemotherapeutics or antibiotics). The medical device can also be, for example, tubes, multilumen tubes, sutures, non-wovens, meshes, wires including wires with plastic shells or coatings, hydrocephalus shunts and other neuroimplants, implantable drains, valves and ports, foams, microspheres and nanospheres.

The medical devices can leave a gentian violet stain on the skin of a subject and/or along the track of the device within the subject. The stain can be decolored by a method comprising applying peroxide to the stain and treating the peroxide with non-ionized metal, such as silver nanoparticles. Preferably, the molar ratio of gentian violet:silver is less than 1:1. The invention thus provides kits including any of the implantable medical devices disclosed herein and non-ionized metal particles for use in decoloring a gentian violet stain produced by the device. Examples of non-ionized metals include, but are not limited to, silver, gold, platinum, copper, iron and zinc. Silver is a preferred non-ionized metal. Nanoparticles are a preferred type of particle. Silver nanoparticles are most preferred. The kit can further comprise a peroxide, such as for example, hydrogen peroxide, inorganic peroxides (magnesium, calcium, and barium peroxide), peroxyacids, peresters, benzoyl peroxide, acetone peroxide, and methyl ethyl ketone peroxide. Hydrogen peroxide is a preferred peroxide. Kits with an inorganic peroxide, such as magnesium, calcium, or barium peroxide, may also comprise an acid such as sodium bisulfate.

The present invention is illustrated in the following Experimental Details section, which is set forth to aid in the understanding of the invention, and should not be construed to limit in any way the scope of the invention as defined in the claims that follow thereafter.

### EXPERIMENTAL DETAILS

### Examples I-II

### Incorporation of Gentian Violet into Tecothane®-2095A Resin

Experiments were performed to incorporate gentian violet into Tecothane®-2095A resin by compounding and extrusion processes. In these examples gentian violet was coated on Tecothane®-2095A pellets by soaking the resin in gentian violet/ethanol mixture and the solvent was evaporated off at ambient conditions. The gentian violet coated pellets were then fed into an extruder or compounder to making tubing or strand pelletized pellets. The gentian violet could also have been fed as a powder directly with the polymer resin for compounding and extrusion. Surprisingly, much higher loadings of gentian violet could be achieved using the high temperature process disclosed herein than had been previously disclosed without degradation of the chemical structure of the gentian violet.

### Example 1

### Compound Tecothane®-2095A Resin with 0.5% Gentian Violet

5 g of gentian violet (Sciencelab, Houston, Tex.) was dissolved in 250 ml 99% ethanol (Sigma-Aldrich, St. Louis, Mo.). 1000 g of Tecothane® 2095A (Noveon, Cleveland, Ohio) resin was added in to the gentian violet/ethanol solution. The ethanol solvent was evaporated off in a chemical fume hood overnight at ambient conditions. The gentian violet coated pellets were then dried at 50° C. and 30 inches Hg for 24 hrs prior to compounding.

The dried gentian violet coated resins were starve-fed into a 18 mm Leistritz intermeshing twin screw extruder (Somerville, N.J.) from a K-tron feeder (Pitman, N.J.) at a rate of 2.5 kg/hr. The extruder was set at 231 rpm for screw speed and the barrel zone temperatures were set from 329° F. (165° C.) thru 338° F. (170° C.). The extrudate was pelletized into small pellets.

### Example 2

### 2% Gentian Violet Loaded Tecothane® Tube

20 g of gentian violet (Sigma-Aldrich, St. Louis, Mo.) was dissolved in 1000 ml ethanol (Sigma-Aldrich, St. Louis, Mo.). 1000 g of Tecothane® resin was added into the gentian violet/ethanol solution. The ethanol solvent was evaporated off in the chemical fume hood overnight at ambient conditions. The gentian violet coated resin then dried at 65° C. and 30 inches Hg for 4 hrs prior to compounding.

The dried gentian violet coated resins were gravity fed into a ⅝′ Randcastle single screw (Cedar Grove, N.J.) microextruder. The microextruder was set at 20 rpm for screw speed and barrel zone temperatures were set from 360° F. thru 375° F. A 5 Fr tubing was drawn from a BH25 tooling (San Marcos, Calif.).

### Characterization of Gentian Violet Compounded in Tecothane® Via HPLC

Gentian violet contents from compounded resin and tube sample were analyzed via HPLC method. HPLC analysis on GV loaded resin or compounded pellets was performed by weighing 0.1-0.3 g, which is roughly equivalent to 10-20 pellets (depending on the weight of the raw material used) of each formulation (n=3) and digesting in THF (5 mL or 7.5 mL) in 50 mL centrifuge tubes. Samples were allowed to sit for 45 minutes, and then vortexed until all of the polymer dissolved. An equal amount of deionized (DI) water was then added (5 or 7.5 mL), and the samples were again vortexed for 5 minutes, and then centrifuged for 10 minutes. A portion of each sample was then added to an HPLC vial and capped prior to HPLC analysis.

HPLC analysis on tubing was performed by cutting and measuring 1 cm segments of each formulation (n=3) and digesting in THF (10 mL or 20 mL) in 50 mL centrifuge tubes. Samples were allowed to sit for 45 minutes, and then vortexed for 5 minutes, or until none of the polymer was stuck to the bottom of the tubes. An equal amount of deionized (DI) water was then added (10 or 20 mL), and the samples were again vortexed for 5 minutes, and then centrifuged for 10 minutes. A portion of each sample was then added to an HPLC vial and capped prior to HPLC analysis.

HPLC analysis was performed on an Agilent 1200 Series LC using an Agilent Eclipse XDB-CN 5μ 4.6×150 mm column with the corresponding guard column. A gradient program was run using two solvent reservoirs:

MP A: 100% DI Water/0.2% Trifluoroacetic Acid,

MP B: 100% Acetonitrile/0.2% Trifluoroacetic Acid.

### Program: 0-5 min 35% B, 5-6 min 35-40% B, 6-12 min 40% B, 12-16 min 35% B.

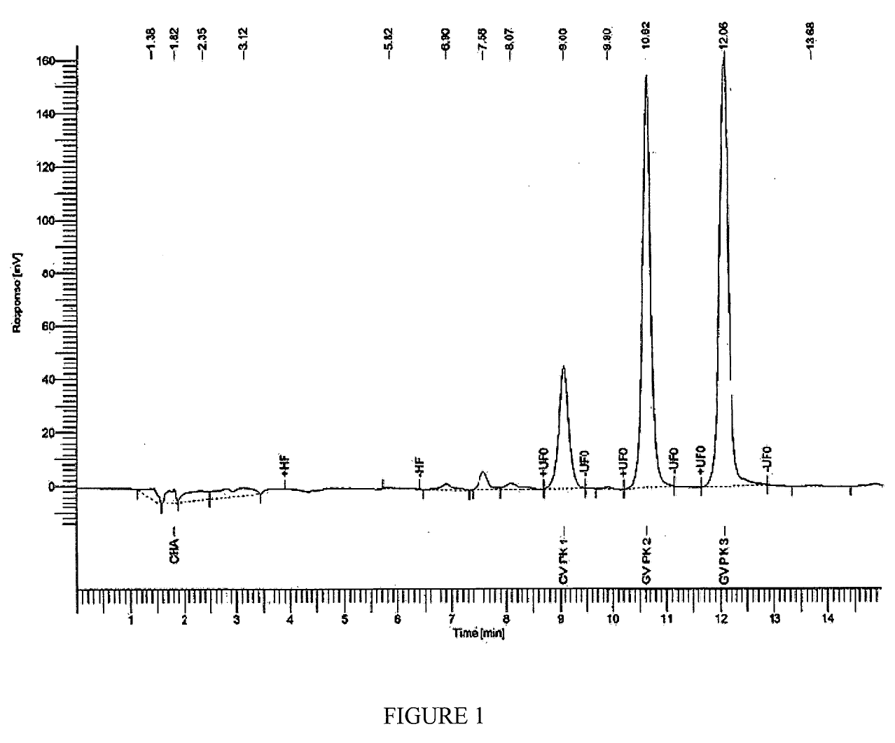

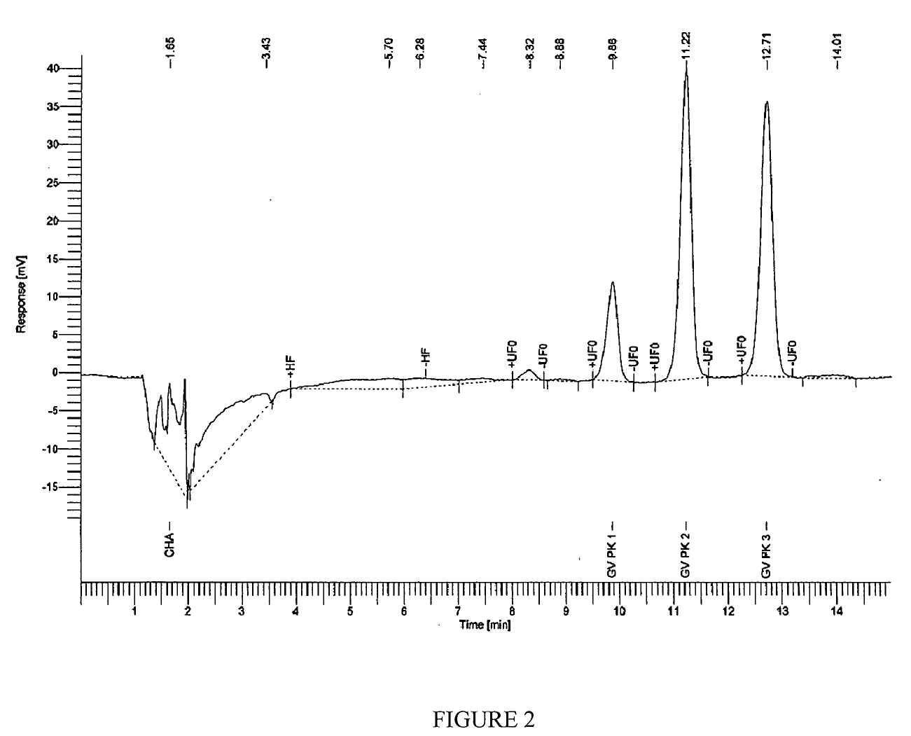

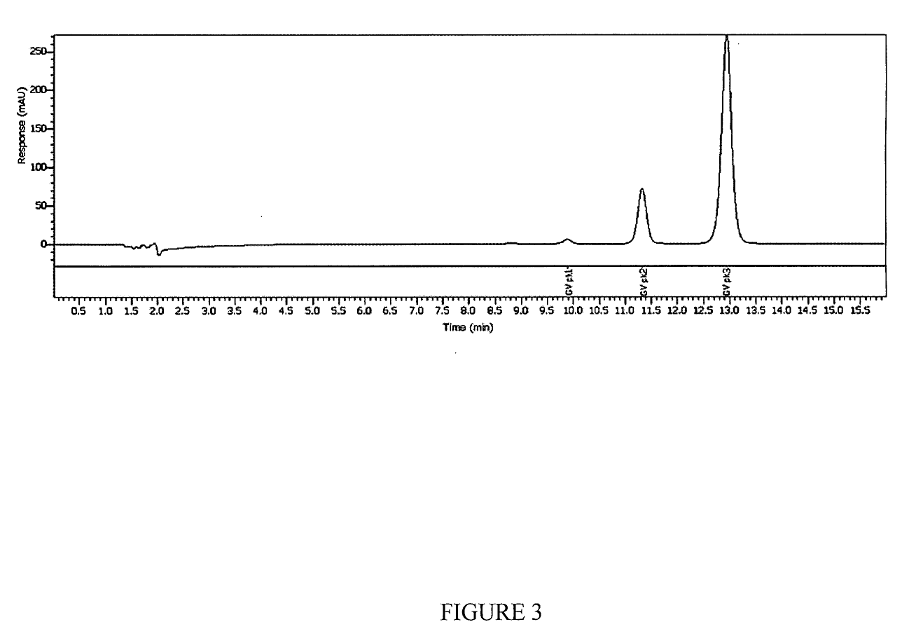

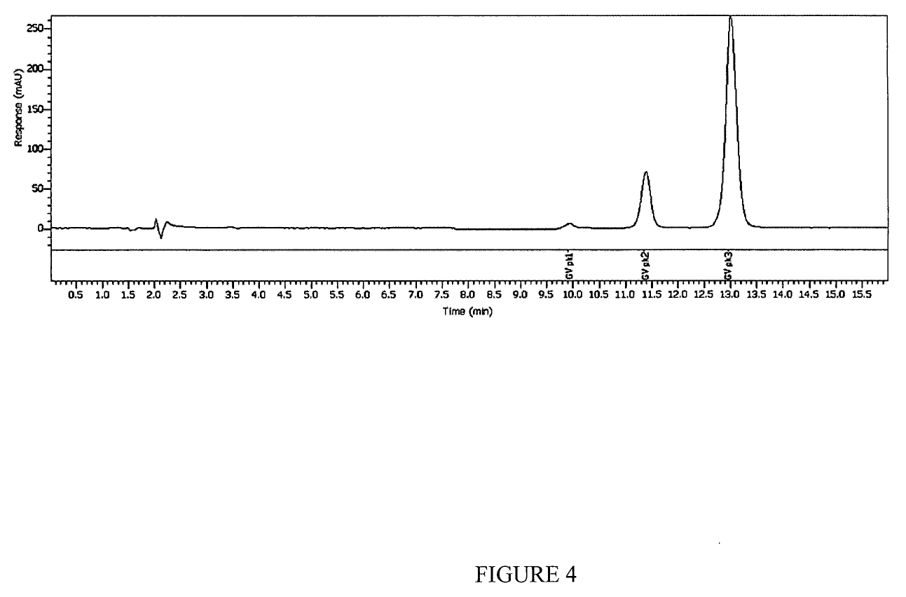

The gentian violet content of the compounded resin and extruded tube is shown in Table 1. Table 2 shows a comparison of the relative peak areas of gentian violet peaks of standard vs. sample. Percent area is based only on the total area of the three peaks, and not any other peaks shown in the chromatograms, which are shown in FIG. 1 and FIG. 2 for standard and sample (Sciencelab), respectively. Table 3 shows a comparison of the relative peak areas of gentian violet peaks of standard vs. sample (from Aldrich). FIG. 3 and FIG. 4 show the chromatographs of standard (Aldrich) and tube sample, respectively.

TABLE 1
GV content of compounded resin and extruded tube.

| | Theoretical | Measured GV |
|---|---|---|
| | loading (% | loading (% |
| Sample | w/w) | w/w) via HPLC |
| Compounded | 0.5 | 0.67% |
| Resin | | |
| Tube | 2.0 | 2.2% |

TABLE 2
Relative % Areas of each peak, Standard vs Compounded sample.

| | Standard | | Sample | |
|---|---|---|---|---|
| | | Relative | | Relative % |
| Peak # | Area (uV-s) | % area | Area (uV-s) | area |
| Peak 1 | 640917.67 | 14.75% | 191846.97 | 14.22% |
| Peak 2 | 1789022.3 | 41.17% | 583737.5 | 43.28% |
| Peak 3 | 1915778.8 | 44.08% | 573305.43 | 42.50% |
| Total area | 4345718.8 | | 1348889.9 | |

TABLE 3
Relative % Peak Area, Standard vs tube sample.

| | % Relative Peak area | | |
|---|---|---|---|
| | Peak 1 | Peak 2 | Peak 3 |
| Tube sample | 1.51 | 18.27 | 80.21 |
| Standard | 1.93 | 19.47 | 78.59 |

### Example III

### Temperature Effect on Degradation of Extruded Gentian Violet. Gentian Violet in Tecothane® Extrusions w/30% Filler

Extruded gentian violet was found to degrade at a processing temperature of 217° C. (starting at about 209° C.); however, processing under about 207° C. does not degrade gentian violet. One of the surprising findings about the temperature effects is that certain classes of polyurethanes are processable below the degradation temperature and other classes are not.

Compounding Conditions for stable gentian violet:

Barrel Temperatures:
Zone 1-5=170° C.
Zone 6=175° C.,

Melt Temp=194° C.,

Screw Speed=100 RPM,

Pressure=˜180 bar.

Compounding Temperature for degraded gentian violet:

Zone 1-6=217° C.

Compounding was performed with Sigma Aldrich ACS grade gentian violet, lot 016K3691. HPLC analysis was performed by cutting and measuring 1 cm segments of each formulation (n=3) using the method described in Examples I and II.

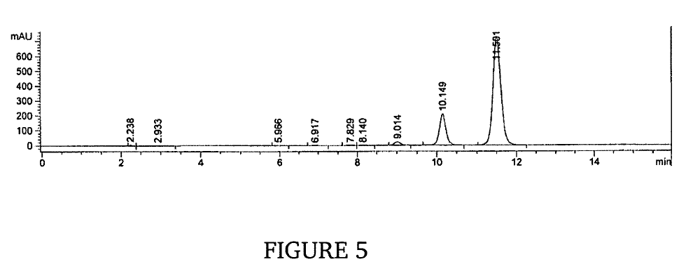

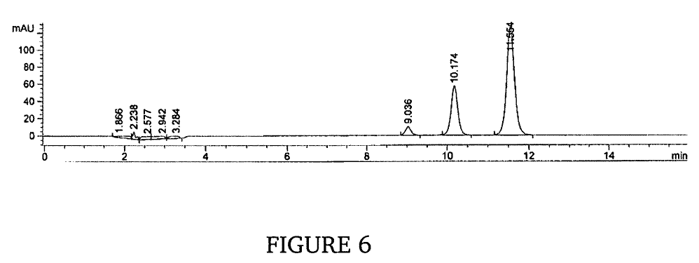

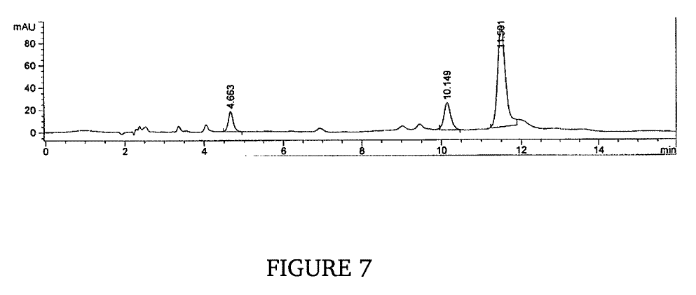

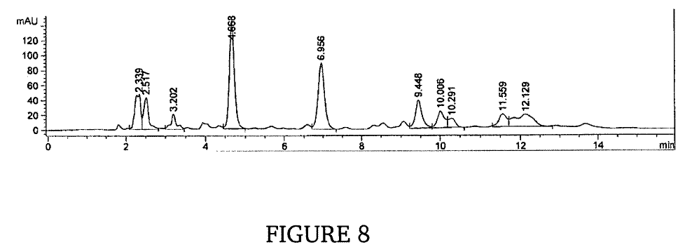

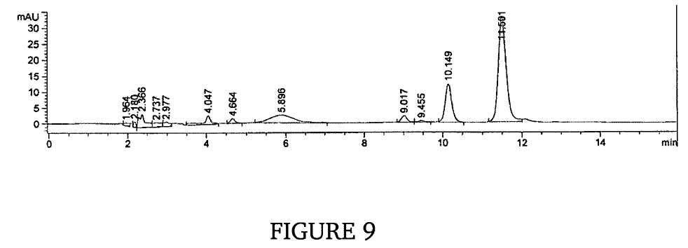

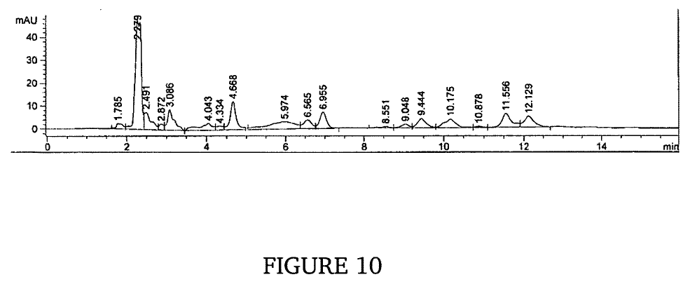

The chromatograms in FIGS. 5-10 show the stable gentian violet followed by the degraded gentian violet, at 588 nm, 253 nm, and 280 nm. The degradation peaks from the 217° C. sample are evident as the new peaks at 253 and 280 nm wavelengths. The chromatogram of the low processing temperature was identical to that of the Gentian Violet raw material.

### Example IV

### Temperature Effect (196° C.-219° C.) on Degradation of Extruded Gentian Violet. Compounded Carbothane® Resin with 0.5% GV

5 g of gentian violet (Sigma-Aldrich, St. Louis, Mo.) was dissolved in 250 ml 99% ethanol (Sigma-Aldrich, St. Louis, Mo.). 1000 g of Carbothane®-3585A-B20 (Noveon, Cleveland, Ohio) resin was added in to the gentian violet/ethanol solution. The ethanol solvent was evaporated off in a chemical fume hood overnight at ambient conditions. The gentian violet coated pellets were then dried at 50° C. and 30 inches Hg for 24 hrs prior to compounding.

The dried gentian violet coated resins were starve-fed into a 18 mm Leistritz intermeshing twin screw extruder (Somerville, N.J.) from a K-Tron feeder (Pitman, N.J.) at a rate of 2.5 kg/hr. The heating profile for each run was varied. The melt temperatures from each run are recorded in Table 4.

HPLC analysis was performed by selecting several pellets of each formulation (n=3) using the method described in Examples I and II.

GV content is essentially unchanged up to a processing temperature of 206° C. The onset of degradation appears at 209° C. and becomes worse at 219° C.

TABLE 4
GV Contents from compounded Carbothane ® at
moderate temperatures.

| | | GV measured from | GV measured from |
|---|---|---|---|
| | Melt Temp | unprocessed resin | compounded resin |
| Sample # | (° C.) | (% w/w) | (% w/w) via HPLC |
| GV control | | 0.50 | |
| 3 | 196 | | 0.49 |
| 4 | 206 | | 0.51 |
| 5 | 209 | | 0.43 |
| 6 | 219 | | 0.40 |

### Example V

### Temperature Effect on Degradation of Extruded Gentian Violet—High Temperatures. Compounded Carbothane® Resin with 0.5% GV

The sample preparation for loading GV on resin pellets is similar to EXAMPLE IV. Gentian violet was purchased from (Sigma-Aldrich, St. Louis, Mo.) and Carbothane®-3585A-B20 (Noveon, Cleveland, Ohio) was used. The heating profile was varied for each run. The melt temperatures are showed in Table 5.

There is significant degradation of the GV above 220° C. as evidenced by the dramatic drop in extractable GV content.

TABLE 5
GV contents from compounded Carbothane ® at high temperatures.

| | | GV measured from | GV measured from |
|---|---|---|---|
| | Melt Temp | unprocessed resin | compounded resin (% |
| Sample # | (° C.) | (% w/w) | w/w) via HPLC |
| GV control | | 0.45 | |
| 7 | 188 | | 0.42 |
| 8 | 221 | | 0.15 |
| 9 | 230 | | 0..13 |
| 10 | 235 | | 0.11 |
| 11 | 241 | | 0.09 |
| 12 | 246 | | 0.06 |

### Example VI

### Dermal Decolorization of Gentian Violet Using Hydroxyl Radicals

### Overview

Gentian violet (GV) can leech out from a medical device such as a catheter, leaving a dermal stain around the site of contact of the device with the skin. A means of removing this stain, after removal of the device, can provide a cosmetic benefit. Decolorization of dye effluents has acquired increasing attention within the textile industry since dye wastewater pollutants are sources of environmental pollution (Roxon et al. 1967). Physical and chemical decolorization of GV has been investigated (Saquib and Muneer 2003). It has been reported that decolorization of GV was achieved by oxidation.

In the present example, the use of hydrogen peroxide (H₂O₂) as the oxidizing species was investigated but the method of activating it was altered. The decolorization of gentian violet by H₂O₂ was investigated by three different activation techniques: photolytic split of H₂O₂ (by UV light), decomposition of H₂O₂ by non-ionic silver (Ag) nanoparticles, and decomposition of H₂O₂ by ascorbic acid (Vitamin-C). It was determined that certain methods of activation are able to convert the GV to a colorless (oxidized) form.

### Materials/Methods

The following material were used: Crystal Violet ACS (lot #036K0709, Sigma-Aldridge) (Gentian violet and Crystal violet are interchangeable names of the same compound), Hydrogen Peroxide (30% ACS lot #060350, Fisher Scientific), Nano-particle Silver, Ascorbic Acid, and TRIS (Hydroxymethyl Aminomethane) (lot #062202, Fisher BioReagents).

The following three protocols were evaluated:

Protocol 1. Photo-oxidation of GV with H₂O₂
1.1. 0.15 g GV (0.1% w/v) was dissolved in 150 ml 3% H₂O₂. The solution was exposed to UV light (Bio-lab hood) for 2 hours. 5 ml of solution was extracted out, in increments of 5 mins.
1.2. 10 μl of 0.1% GV in DI water was spiked onto a plated agar plate. After staining of the agar plate (evaporation of most of the water from the spiked drop), H₂O₂ was pipetted onto the stain in increments of 50 μl every 5 mins for 2 hours, under the UV light.
   Protocol 2. Oxidation of GV by activation of H₂O₂ with Ag
2.1. 10.6 mg of nano-particle Silver was mixed into 40 ml 3% H₂O₂ with constant stirring. 0.1% GV in water was added at various increments (10 μl-1 ml) until purple color persisted. Molar concentration ratios (GV:Ag) were calculated to determine the molar ratio where GV is no longer oxidized.
2.2 10 μl of 0.1% GV in DI water was spiked onto a plated agar plate. After staining of the agar plate (evaporation of most of the water from the spiked drop), 0.009 g of Ag was applied to the surface of the stain and 100 μl of H₂O₂ was pipette onto the Ag.
   Protocol 3. Oxidation of GV by activation of H₂O₂ with Vitamin C 1% Vitamin C (w/v) was dissolved in 100 ml 3% H₂O₂. 7 g of TRIS buffer was also dissolved in the H₂O₂. 0.1% GV in water was added at increments of 10 μl until the purple color persisted.

### Results and Discussion

Protocol 1. No color change was observed in all test samples treated with UV light. Solution and agar stain remained purple after numerous hours under UV light. Since it has been reported that H₂O₂ splits photolytically to produce OH (Peyton and Glaze 1988), the intensity of the UV light used here might not have been adequate to generate these radicals.

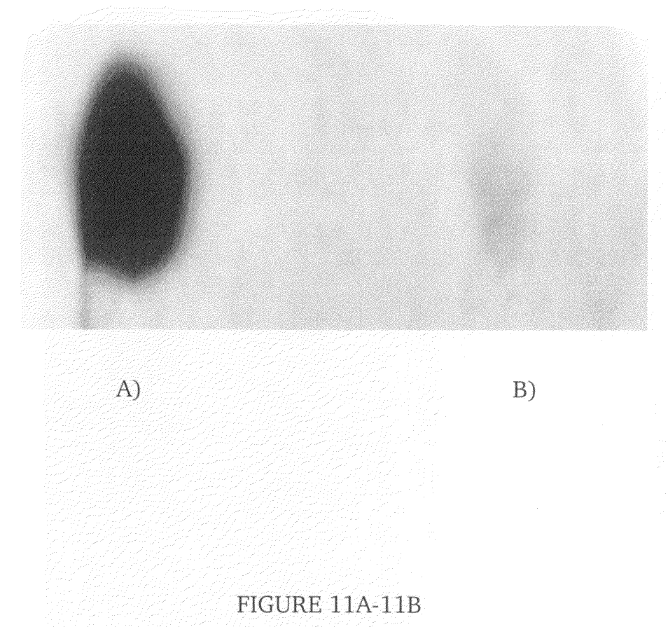

Protocol 2. As the 0.1% GV solution was pipetted in the Ag+H₂O₂ solution, the purple color was observed to fade to a faint yellow color. This faint yellow color became darker as more GV was added, until the purple color remained permanently. The molar ratio of GV:Ag at this point was calculated to be 1:1. Similarly, the purple stain on the agar was observed to turn to a faint yellow color after 10 mins (FIG. 11B).

Protocol 3. The addition of Vitamin C to H₂O₂ was observed to reduce the pH of the solution. TRIS buffer was added to neutralize the acidic solution to a pH range of 6-7. With the addition of the first 10 μl increment of 0.1% GV, the solution turned and remained purple.

In all test techniques, only with the use of non-ionic silver was hydrogen peroxide able to neutralize the purple color from GV (from purple to faint yellow). Neither vitamin C nor UV light was a strong enough activating agent, at the concentrations tested here.

### REFERENCES

Hanna H, P Bahna, R Reitzel, T Dvorak, G Chaiban, R Hachem and I Raad. Comparative in vitro efficacies and antimicrobial durabilities of novel antimicrobial central venous catheters. Antimicrob. Agents Chemother. 50: 3283-3288, 2006.
Harter C, H J Salwender, A Bach, G Egerer, H Goldschmidt and A D Ho. Catheter-related infection and thrombosis of the internal jugular vein in hematologic-oncologic patients undergoing chemotherapy: a prospective comparison of silver-coated and uncoated catheters. Cancer 94: 245-251, 2002.
Peyton G R, Glaze W H. Destruction of pollutant in water with ozone in contamination with ultraviolet radiation. Environ Sci Technol 1988; 22:761-7.
Rooden C J, E F Schippers, R M Y Barge, F R Rosendaal, H F L Guiot, F J. M. van der Meer, A E Meinders, M V Huisman. Infectious complications of central venous catheters increase the risk of catheter-related thrombosis in hematology patients: A prospective study. Journal of Clinical Oncology 23: 2655-2660, 2005.
Roxon J J, Ryan A J, Wright S E. Reduction of water-soluble azo dyes by intestinal bacteria. Food Cosmet Toxicol 1967; 5: 367-9.
Safdar N, Maki D G. The pathogenesis of catheter-related bloodstream infection with noncuffed short-term central venous catheters. Intensive Care Med. 30(1):62-7, 2004, Epub 2003 November 26.
Saint S, Veenstra D L, Lipsky B A. The clinical and economic consequences of nosocomial central venous catheter-related infection: are antimicrobial catheters useful? Infect Control Hosp Epidemiol. 21(6):375-80, 2000.
Saquib, M. Muneer, M. TiO₂-mediated photocatalytic degradation of a triphenylmethane dye (gentian violet), in aqueous suspensions. Dyes and Pigments 56 (2003) 37-49.
U.S. Patent Application Publication No. US 2003/0078242 A1, published Apr. 24, 2003, Raad et al. Novel antiseptic derivatives with broad spectrum antimicrobial activity for the impregnation of surfaces.
U.S. Patent Application Publication No. US 2005/0131356 A1, published Jun. 16, 2005, Ash et al. Medical devices exhibiting antibacterial properties.
U.S. Patent Application Publication No. US 2005/0197634 A1, published Sep. 8, 2005, Raad et al. Methods for coating an impregnating medical devices with antiseptic compositions.
U.S. Pat. No. 5,451,424, issued Sep. 19, 1995, Solomon et al., Anti-infective and antithrombogenic medical articles and method for their preparation.
U.S. Pat. No. 5,688,516, issued Nov. 18, 1997. Raad et al. Non-glycopeptide antimicrobial agents in combination with an anticoagulant, an antithrombotic or a chelating agent, and their uses in, for example, the preparation of medical devices.
U.S. Pat. No. 5,707,366, issued Jan. 13, 1998, Solomon et al., Anti-infective and antithrombogenic medical articles and method for their preparation.
U.S. Pat. No. 5,709,672, issued Jan. 20, 1998, Illner, Silastic and polymer-based catheters with improved antimicrobial/antifungal properties.
U.S. Pat. No. 6,261,271, issued Jul. 17, 2001, Solomon et al., Anti-infective and antithrombogenic medical articles and method for their preparation.
U.S. Pat. No. 6,273,875 B1, issued Aug. 14, 2001. Siman et al. Medical devices having improved antimicrobial/antithrombogenic properties.
U.S. Pat. No. 6,528,107 B2, issued Mar. 4, 2003. Chinn et al. Method of producing antimicrobial and antithrombogenic medical devices.

1. A method of preparing an implantable medical device treated with gentian violet, the method comprising extruding gentian violet with a thermoplastic at a temperature below 207° C., where the concentration of gentian violet is 0.05% to 20% by weight of the thermoplastic, to produce a gentian violet-treated thermoplastic implantable medical device.

2. The method of claim 1, wherein the concentration of gentian violet is greater than 1% by weight.

3. The method of claim 1, wherein the processing temperature is below 200° C.

4. The method of claim 1, wherein thermoplastic resin is coated with gentian violet prior to extrusion.

5. The method of claim 1, wherein thermoplastic resin and gentian violet powder are compounded prior to extrusion.

6. The method of claim 1, wherein the thermoplastic is a polyurethane, a polyolefin, a vinyl polymer, an acrylate polymer, a polyamide, a polyester, a fluoroelastomer, or a copolymer or a blend thereof.

7. (canceled)

8. The method claim 1, wherein the gentian violet-treated thermoplastic is co-extruded with another antimicrobial agent.

9. The method of claim 1, wherein the implantable medical device is a catheter.

10. The method of claim 9, wherein the catheter is for implantation in a blood vessel or a body cavity.

11. (canceled)

12. The method of claim 9, wherein the catheter is a venous catheter, an arterial catheter, a dialysis catheter, a urinary catheter or a tracheal catheter or tracheal tube.

13. An implantable medical device prepared by the method of claim 1.

14. An implantable medical device comprising thermoplastic treated with gentian violet, wherein the thermoplastic has a processing temperature below 207° C. and wherein the concentration of gentian violet in the device is 0.05% to 20% by weight of the thermoplastic.

15. The implantable medical device of claim 14, wherein the device is a laminated implantable medical device comprising a thermoplastic layer treated with gentian violet, wherein the thermoplastic in the layer has a processing temperature below 207° C. and wherein the concentration of gentian violet in the layer is 0.05% to 20% by weight of the thermoplastic in the layer.

16. The implantable medical device of claim 14, wherein the thermoplastic in the layer is a polyurethane, a polyolefin, a vinyl polymer, an acrylate polymer, a polyamide, a polyester, a fluoroelastomer, or a copolymer or a blend thereof.

17. (canceled)

18. The implantable medical device of claim 14, wherein the implantable medical device is a catheter.

19. An implantable medical device comprising a thermoplastic treated with gentian violet or a thermoset treated with gentian violet, wherein the concentration of gentian violet in the device is greater than 1% by weight of the thermoplastic or the thermoset.

20. The implantable medical device of claim 19, wherein the device is a laminated implantable medical device comprising a thermoplastic layer treated with gentian violet or a thermoset layer treated with gentian violet, wherein the concentration of gentian violet in the layer is greater than 1% by weight of the thermoplastic in the layer or the thermoset in the layer.

21. (canceled)

22. The device of claim 19, wherein the concentration of gentian violet is greater than 1.1%, 1.5%, 2%, 2.5%, 3%, 4%, 5%, 7.5% or 10% by weight of the thermoplastic or the thermoset.

23. The device of claim 19, wherein the concentration of gentian violet is up to 20% by weight of the thermoplastic or the thermoset.

24-26. (canceled)

27. The device of claim 19, wherein the thermoplastic has a processing temperature below 207° C.

28. The device of claim 19, wherein the thermoplastic is a polyurethane, a polyolefin, a vinyl polymer, an acrylate polymer, a polyamide, a polyester, a fluoroelastomer, or a copolymer or a blend thereof.

29-33. (canceled)

34. The device of claim 19, wherein device comprises a plurality of regions and gentian violet is present in less than all of the regions.

35. The device of claim 19, wherein the device is a catheter.

36. The catheter of claim 35, wherein the catheter comprises a tip region and gentian violet is present in the tip region.

37. The catheter of claim 35, wherein the catheter comprises a proximal hub region and gentian violet is present in the hub region.

38-40. (canceled)

41. A kit comprising the implantable medical device of claim 35 and non-ionized metal particles for use in decoloring a gentian violet stain produced by the device.

42-47. (canceled)
